SEQUENCE LISTING (2 sequences)

SEQ ID NO: 1 - DNA (other nucleic acid), 39 bp, not provided; double-stranded, linear
  description: /desc = “Oligonucleotide”
ccctcgaggccatcgatgaggcctgactttttcaataaa

SEQ ID NO: 2 - DNA (other nucleic acid), 33 bp, not provided; double-stranded, linear
  description: /desc = “Oligonucleotide”
gcgtcgacagctcagtcccaggcctcagacgag

(19) United States
(12) Patent Application Publication
(10) Pub. No.: US 2001/0014319 A1
(43) Pub. Date: Aug. 16, 2001

(54) RECOMBINANT VIRUSES EXPRESSING LECITHIN-CHOLESTEROL ACYLTRANSFERASE, AND USES THEREOF IN GENE THERAPY

(75) Inventors: PATRICE DENEFLE, SAINT MAUR (FR); NICOLAS DUVERGER, PARIS (FR); MARTINE LATTA-MAHIEU, CHARENTON LE PONT (FR); SANDRINE SEGURET, MONTIGINY LE BRETONNEUX (DE)

Correspondence Address: FINNEGAN HENDERSON FARABOW GARRETT &, DUNNER, LLP, 1300 I STREET, NW, WASHINGTON,, DC, 200053315

(73) Assignee: RHONE-POULENC RORER S.A.

(21) Appl. No.: 08/913,699

(22) PCT Filed: Mar. 12, 1996
(86) PCT No.: PCT/FR96/00381

§ 371 Date: None

(30) Foreign Application Priority Data
Mar. 14, 1995 (FR) 95/02943

(51) Int. Cl. A61K 48/00; C07H 21/04

(52) U.S. Cl. 424/93.1; 514/44; 424/93.2; 424/93.21; 435/456; 435/320.1; 536/23.1; 536/23.2; 536/23.5

(57) ABSTRACT

Defective recombinant viruses containing an inserted gene coding for all or part of lecithin-cholesterol acyltransferase (LCAT) or a variant thereof, pharmaceutical compositions containing said viruses, and the use thereof for treating or preventing dyslipoproteinaemia-related diseases, are disclosed.

The present invention relates to new recombinant viruses, to their preparation and their use in gene therapy, for the transfer and expression in vivo of desired genes. More precisely, it relates to new recombinant viruses comprising an inserted gene encoding all or part of lecithin-cholesterol acyltransferase (LCAT) or a variant thereof. The present invention also relates to pharmaceutical compositions comprising the said recombinant viruses. More particularly, the present invention relates to defective recombinant viruses and their use for the prevention or the treatment of pathologies linked to dyslipoproteinaemias, which are known for their serious consequences at the cardiovascular and neurological level.

Dyslipoproteinaemias are disorders of the metabolism of the lipoproteins responsible for the transport, in the blood and peripheral fluids, of lipids such as cholesterol and triglycerides. They result in major pathologies, linked respectively to hypercholesterolemia or hypertriglyceridemia, such as especially atherosclerosis. Atherosclerosis is a polygenic complex disease which is defined from the histological point of view by deposits (lipid or fibrolipid plaques) of lipids and of other blood derivatives in the wall of the large arteries (aorta, coronary arteries, carotid). These plaques, which are calcified to a greater or lesser extent according to the progression of the process, can be associated with lesions and are linked to the accumulation, in the arteries, of fatty deposits consisting essentially of cholesterol esters. These plaques are accompanied by a thickening of the arterial wall, with hypertrophy of the smooth muscle, the appearance of spumous cells and the accumulation of fibrous tissue. The atheromatous plaque is very clearly in relief on the wall, which confers on it a stenosing character responsible for vascular occlusions by atheroma, thrombosis or embolism which occur in the patients most affected. Hypercholesterolemias can therefore result in very serious cardiovascular pathologies such as infarction, sudden death, cardiac decompensation, cerebral vascular accidents and the like.

It is therefore particularly important to be able to have available treatments which make it possible to reduce, in certain pathological situations, the plasma cholesterol levels or even to stimulate the efflux of cholesterol (reverse transport of the cholesterol) in the peripheral tissues in order to discharge the cells having accumulated cholesterol within the context of the formation of an atheroma plaque. The cholesterol is carried in the blood by various lipoproteins including the low-density lipoproteins (LDL) and the high-density lipoproteins (HDL). The LDLs are synthesized in the liver and make it possible to supply the peripheral tissues with cholesterol. In contrast, the HDLs capture cholesterol in the peripheral tissues and transport it to the liver where it is stored and/or degraded.

At present, dyslipemias and in particular hypercholesterolemias are treated essentially by means of compounds which act either on the biosynthesis of cholesterol (inhibitors of hydroxymethylglutaryl-coenzymeA reductase, statins), or on the capture and elimination of bile cholesterol (sequestrants or resins), or alternatively on lipolysis by a mode of action which remains to be elucidated from the molecular point of view (fibrates). Consequently, all the major categories of drugs which have been used in this indication (sequestrants, fibrates or statins), are designed only for the preventive aspect of the formation of the atheroma plaque and not in fact for the treatment of the atheroma. The current treatment for atheroma, following a coronary accident, are only palliative since they do not act on cholesterol homeostasis and they are surgical acts (coronary by-pass, angioplasty).

A first approach for the treatment of these pathologies by gene therapy has been described in Application W094/25073. This approach is based, in particular, on the direct transfer of genes encoding apolipoproteins. The present invention constitutes a new therapeutic approach for the treatment of pathologies linked to dyslipoproteinaemias. It is based more particularly on the transfer of genes encoding enzymes involved in the catabolism of cholesterol. In particular, the transfer and the expression in vivo of the LCAT according to the invention makes it possible, advantageously, to act not only on the circulating HDL levels, but also on their enzymatic activity linked to the reverse transport of cholesterol. This approach therefore has a double stimulating effect aimed at bringing cholesterol back to the liver. The present invention is also based on the use of viruses which make it possible to transfer and to express genes encoding enzymes of the metabolism of cholesterol in the liver, and to secrete the said enzymes into the circulatory system where they exert their activity with a high efficiency. The examples presented later indicate especially that adenoviruses are capable, depending on the mode of administration, of transferring and of expressing efficiently, for a long period and without cytopathologic effect, the gene expressing lecithin-cholesterol acyltransferase (LCAT).

A first subject of the invention therefore consists in a defective recombinant virus containing at least one inserted gene encoding all or part of lecithin-cholesterol acyltransferase (LCAT) or a variant thereof.

The subject of the invention is also the use of such a defective recombinant virus for the preparation of a pharmaceutical composition intended for the treatment or for the prevention of pathologies linked to dyslipoproteinaemias.

Human lecithin-cholesterol acyltransferase (LCAT) is a glycosylated protein of 416 amino acids having a relative molecular mass of 65 to 69 kD. The gene, as well as the cDNA, encoding LCAT, 4200 and 1744 bp in length respectively, have been cloned and sequenced (McLean et al., Proc.Natl.Acad Sci.83 (1986) 2335 and McLean et al., Nucleic Acids Res. 14(23) (1986) 9397). LCAT is an enzyme which catalyses the esterification of free cholesterol by the transfer of an acyl group from phosphatidylcholine onto a hydroxyl residue of the cholesterol, with formation of cholesterol ester and lysophosphatidylcholine. It is synthesized in man specifically in the liver and it is released into the plasma (6 μg/ml), where it is combined with high-density lipoproteins (HDL), termed anti-atherogenic lipoproteins. These particles possess the capacity to accept the cholesterol which exists in excess in the cells, which is then esterified by LCAT. The HDLs which are high in cholesterol esters are captured by the liver and then eliminated therein. This mechanism, which allows the removal of excess cholesterol from the body, is called reverse cholesterol transport and is clearly involved in the prevention of atherogenesis (Ana Jonas BBA 1084 (1991) 273 and Johnson et al. BBA 1085 (1991)205). LCAT, by creating a gradient of free cholesterol between the plasma membranes and the circulating lipoproteins, probably plays a major role in this process.

The physiological consequences of a partial or total absence of activity of the LCAT enzyme in the plasma are illustrated by the pathological changes observed in the “Fish Eye Disease” (FES) syndrome and the conventional LCAT deficiency syndrome. The clinical symptoms of FES are the opacity of the cornea as well as a renal impairment and an anaemia. These two syndromes are associated with a hypoalphalipoproteinaemia and an increase in the plasma triglycerides. They can be distinguished by the biochemical assay of the LCAT activity in the plasma. No plasma cholesterol esterification activity is detectable in a patient suffering from conventional LCAT deficiency whereas in a patient having an FES profile, a residual LCAT activity is observed. The transfer of an LCAT gene according to the invention constitutes a new approach for the treatment of cardiovascular pathologies. The capacity to transfer this gene and to overexpress LCAT in vivo makes it possible, according to the invention, to exert a double stimulation activity on the efflux of cholesterol, linked on the one hand to the increase in the level of circulating HDLs and, on the other hand, to the increase in the enzymatic activity of these HDLs.

In the viruses of the invention, the inserted gene may be a complementary DNA fragment (cDNA), genomic DNA (gDNA), or a hybrid construct consisting for example of a cDNA into which would be inserted one or more introns. It may also be synthetic or semisynthetic sequences. As indicated above, it may be a gene encoding all or part of LCAT or of a variant thereof. For the purposes of the present invention, the term variant designates any mutant, fragment or peptide having at least one biological property of LCAT, as well as any natural variant of LCAT. These fragments and variants may be obtained by any technique known to persons skilled in the art, and especially by genetic and/or chemical and/or enzymatic modifications, or alternatively by expression cloning, allowing the selection of variants according to their biological activity. The genetic modifications include suppressions, deletions, mutations and the like.

The inserted gene for the purposes of the invention is preferably the gene encoding all or part of the human LCAT. It is more particularly a cDNA or a gDNA.

Generally, the inserted gene also comprises sequences allowing its expression in the infected cell. These may be sequences which are naturally responsible for the expression of the said gene when these sequences are capable of functioning in the infected cell. They may also be sequences of different origin (which are responsible for the expression of other proteins, or even synthetic). In particular, they may be sequences of eukaryotic or viral genes or derived sequences, stimulating or repressing the transcription of a gene in a specific manner or otherwise and in an inducible manner or otherwise. As example, they may be promoter sequences derived from the genome of the cell which it is desired to infect, or from the genome of a virus, and especially the promoters of the adenovirus E1A and MLP genes, the RSV-LTR or CMV promoter, and the like. Among the eukaryotic promoters, there may also be mentioned the ubiquitous promoters (HPRT, vimentin, α-actin, tubulin, and the like), the promoters of the intermediate filaments (desmin, neurofilaments, keratine, GFAP, and the like), the promoters of therapeutic genes (MDR, CFTR, factor VIII type, and the like), the tissue-specific promoters (pyruvate kinase, villin, promoter of the fatty acid-binding intestinal protein, promoter of the a actin of the smooth muscle cells, promoters specific for the liver; Apo AI, Apo AII, human albumin, and the like) or alternatively the promoters which respond to a stimulus (receptor for steroid hormones, receptor for retinoic acid, and the like). In addition, these expression sequences can be modified by addition of activating and regulatory sequences, and the like. Moreover, when the inserted gene does not contain expression sequences, it can be inserted into the genome of the defective virus downstream of such a sequence.

Moreover, the inserted gene generally comprises, upstream of the coding sequence, a signal sequence directing the synthesized polypeptide in the secretory pathways of the target cell. This signal sequence may be the natural signal sequence of LCAT, but it may also be any other functional signal sequence or an artifical signal sequence.

The viruses according to the present invention are defective, that is to say that they are incapable of autonomously replicating in the target cell. Generally, the genome of the defective viruses used within the framework of the present invention therefore lacks at least the sequences necessary for the replication of the said virus in the infected cell. These regions can be either removed (completely or partly), or rendered nonfunctional, or substituted by other sequences and especially by the inserted gene. Preferably, the defective virus nevertheless conserves the sequences in each genome which are necessary for the encapsidation of the viral particles.

The virus according to the invention may be derived from an adenovirus, from an adeno-associated virus (AAV) or from a retrovirus. According to a preferred embodiment, it is an adenovirus.

Various adenovirus serotypes exist, whose structure and properties vary somewhat. Among these serotypes, the use of the type 2 or 5 human adenoviruses (Ad 2 or Ad 5) or of the adenoviruses of animal origin (see application W094/26914) is preferred within the framework of the present invention. Among the adenoviruses of animal origin which can be used within the framework of the present invention, there may be mentioned adenoviruses of canine, bovine, murine (example: MAV1, Beard et al., Virology 75 (1990) 81), ovine, porcine, avian or alternatively simian (example: SAV) origin. Preferably, the adenovirus of animal origin is a canine adenovirus, or more preferably a CAV2 adenovirus [Manhattan strain or A26/61 (ATCC VR-800) for example]. Preferably, adenoviruses of human or canine or mixed origin are used within the framework of the invention.

Preferably, the defective adenoviruses of the invention comprise the ITRs, a sequence allowing the encapsidation and the nucleic acid of interest. Still more preferably, in the genome of the adenoviruses of the invention, at least the E1 region is nonfunctional. The viral gene considered can be rendered non-functional by any technique known to persons skilled in the art, and especially by total suppression, by substitution or partial deletion, or by addition of one or more bases in the gene(s) considered. Such modifications can be obtained in vitro (on the isolated DNA) or in situ, for example by means of genetic engineering techniques, or alternatively by treating with mutagenic agents. Other regions can also be modified, and especially the E3 (W095/02697), E2 (W094/28938), E4 (WO94/28152, W094/12649, W095/02697) and L5 (W095/02697) region. According to a preferred embodiment, the adenovirus according to the invention comprises a deletion in the E1 and E4 regions. According to another preferred embodiment, it comprises a deletion in the E1 region at the level of which the E4 region and the LCAT-encoding sequence are inserted (Cf FR94 13355).

The defective recombinant adenoviruses according to the invention can be prepared by any technique known to persons skilled in the art (Levrero et al., Gene 101 (1991) 195, EP 185 573; Graham, EMBO J. 3 (1984) 2917). In particular, they can be prepared by homologous recombination between an adenovirus and a plasmid carrying, inter alia, the DNA sequence of interest. The homologous recombination occurs after co-transfection of the said adenoviruses and plasmid into an appropriate cell line. The cell line used should preferably (i) be transformable by the said elements, and (ii) contain the sequences capable of complementing the defective adenovirus genome part, preferably in integrated form in order to avoid risks of recombination. As an example of a cell line, there may be mentioned the human embryonic kidney line 293 (Graham et al., J. Gen. Virol. 36 (1977) 59) which contains especially, integrated in its genome, the left hand part of the genome of an Ad5 adenovirus (12%) or lines capable of complementing the El and E4 functions as described especially in applications No. W094/26914 and W095/02697.

Next, the adenoviruses which have multiplied are recovered and purified according to conventional molecular biology techniques as illustrated in the examples.

As regards the adeno-associated viruses (AAV), they are relatively small DNA viruses which become integrated into the genome of the cells which they infect, in a stable and site-specific manner. They are capable of infecting a broad spectrum of cells, without inducing any effect on cell growth, morphology or differentiation. Moreover, they do not seem to be involved in pathologies in man. The genome of the AAVs has been cloned, sequenced and characterized. It comprises about 4700 bases and contains, at each end, an inverted repeat region (ITR) of about 145 bases which serves as replication origin for the virus. The remainder of the genome is divided into 2 essential regions carrying the encapsidation functions: the left hand part of the genome, which contains the rep gene involved in the viral replication and the expression of the viral genes; the right hand part of the genome, which contains the cap gene encoding the virus capsid proteins.

The use of vectors derived from AAVs for the transfer of genes in vitro and in vivo has been described in the literature (see especially WO 91/18088; WO 93/09239; U.S. Pat. No. 4,797,368, U.S. Pat. No. 5,139,941, EP 488 528). These applications describe various constructs derived from AAVs, from which the rep and/or cap genes are deleted and replaced by a gene of interest, and their use for the transfer in vitro (on cells in culture) or in vivo (directly in an organism) of the said gene of interest. The defective recombinant AAVs according to the invention can be prepared by co-transfection, into a cell line infected by a human helper virus (for example an adenovirus), of a plasmid containing the nucleic sequence of interest bordered by two AAV inverted repeat regions (ITR), and of a plasmid carrying the AAV encapsidation genes (rep and cap genes). The recombinant AAVs produced are then purified by conventional techniques. The invention therefore also relates to a recombinant virus derived from the AAVs whose genome comprises an LCAT-encoding sequence bordered by the AAV ITRs. The invention also relates to a plasmid comprising an LCAT-encoding sequence bordered by two ITRs of an AAV. Such a plasmid can be used as it is to transfer the LCAT sequence, optionally incorporated into a liposome vector (pseudo-virus).

As regards the retroviruses, the construction of recombinant vectors has been widely described in the literature: see especially EP 453242, EP 178220, Bernstein et al. Genet. Eng. 7 (1985) 235; McCormick, BioTechnology 3 (1985) .689, and the like. In particular, the retroviruses are integrative viruses which infect dividing cells. The genome of retroviruses essentially comprises two LTRs, an encapsidation sequence and three coding regions (gag, pol and env). In the recombinant vectors derived from retroviruses, the gag, pol and env genes are generally deleted, completely or partly, and replaced by a heterologous nucleic acid sequence of interest. These vectors can be prepared from various types of retroviruses such as especially MoMuLV (murine Moloney leukaemia virus, also called MOMLV), MSV (murine Moloney sarcoma virus), HaSV (Harvey sarcoma virus), SNV (spleen necrosis virus), RSV (Rous sarcoma virus) or alternatively Friend's virus.

To construct the recombinant retroviruses containing an LCAT-encoding sequence according to the invention, a plasmid containing especially the LTRs, the encapsidation sequence and the said coding sequence is generally constructed and then used to transfect a so-called encapsidation cell line capable of providing in trans the retroviral functions which are deficient in the plasmid. Generally, the encapsidation lines are therefore capable of expressing the gag, pol and env genes. Such encapsidation lines have been described in the prior art, and especially the PA317 line (U.S. Pat. No. 4,861,719), the PsiCRIP line (WO 90/02806) and the GP+envAm-12 line (WO 89/07150). Moreover, the recombinant retroviruses may contain modifications in the LTRs so as to suppress the transcriptional activity, as well as extended encapsidation sequences containing a portion of the gag gene (Bender et al., J. Virol. 61 (1987) 1639). The recombinant retroviruses produced are then purified by conventional techniques.

The present invention also relates to a pharmaceutical composition comprising one or more defective recombinant viruses as described above. Such compositions can be formulated for topical, oral, parenteral, intranasal, intravenous, intramuscular, subcutaneous or intraocular administration, and the like.

Preferably, the composition according to the invention contains vehicles pharmaceutically acceptable for an injectable formulation. These may be in particular saline solutions (monosodium or disodium phosphate, sodium, potassium, calcium or magnesium chloride, and the like, or mixtures of such salts), sterile, isotonic, or dry, especially freeze-dried compositions, which, upon addition, depending on the case, of sterilized water or of physiological saline, allow the constitution of injectable solutions.

In their use for the treatment of pathologies linked to dyslipoproteinaemias, the defective recombinant adenoviruses according to the invention can be administered according to various modes, and especially by intravenous injection. Preferably, they are injected at the level of the portal vein. As regards the retroviruses, it may be advantageous to use cells infected ex vivo for their reimplantation in vivo, optionally in the form of neo-organs (WO 94/24298).

The virus doses used for the injection can be adapted according to various parameters, and especially according to the mode of administration used, the relevant pathology or alternatively the desired duration of treatment. In general, the recombinant viruses according to the invention are formulated and administered in the form of doses of between 10⁴ and 10¹⁴ pfu/ml. For the AAVs and the adenoviruses, doses of 10⁶ to 10¹⁰ pfu/ml can also be used. The term pfu (“plaque forming unit”) corresponds to the infectivity of a suspension of virions, and is determined by infection of an appropriate cell culture, and measurement, generally after 48 hours, of the number of plaques of infected cells. The techniques for determining the pfu titre of a viral solution are well documented in the literature.

Moreover, the pharmaceutical compositions of the invention may also contain one or more defective recombinant adenoviruses containing an inserted gene encoding an apolipoprotein. The combination of these two types of genes makes it possible to exert a synergistic effect on the activity of the HDLs and thus on the reverse transport of cholesterol. The adenovirus construct containing an inserted gene encoding an apolipoprotein has been described in application WO 94/25073. A preferred combination comprises an adenovirus according to the invention and an adenovirus containing a gene encoding an apolipoprotein AI or apolipoprotein AIV.

The present invention offers a very efficient new means for the treatment or the prevention of pathologies linked to dyslipoproteinaemias, in particular in the field of cardiovascular conditions such as myocardial infarction, angina, sudden death, cardiac decompensation, cerebrovascular accidents, atherosclerosis or restenosis. More generally, this approach offers a highly promising means of therapeutic procedure for each case where a genetic or metabolic deficiency of LCAT can be corrected.

In addition, this treatment may relate both to man and to any animal such as ovines, bovines, domestic animals (dogs, cats and the like), horses, fish and the like.

The present invention is more fully described with the aid of the examples below, which should be considered as illustrative and non-limiting.

### LEGEND TO THE FIGURES

FIG. 1: Representation of the plasmid pXL2639.

FIG. 2: Representation of the plasmid pXL2640.

FIG. 3: Transfection of the Hep3B cells with an adeno AdCMV hLCAT. The cells Hep3B were infected with an adeno AdCMV hLCAT (open squares) or an adeno AdCMV βgal (filled squares) at multiplicities of infection of 10, 25, 50, 100, 250 and 500. The LCAT activity was measured in the supernatant at 72 h. The determinations were made in duplicate and each value represents the mean±standard deviation.

FIG. 4: Northern-blot analysis of the RNA isolated from the liver of infected or noninfected mice. The total RNA is derived from the livers of the control mice (1), infected with the adeno AdCMV βgal (2) and the adeno AdCMV hLCAT (3). 10 μg of RNA were separated by electrophoresis in formaldehyde-1.2% agarose, transferred onto a nylon membrane and hybridized with various human LCAT and mouse apoE probes.

FIGS. 5A and 5B: Effect of the transfer of the human LCAT gene on the plasma concentrations of total cholesterol and HDL cholesterol. Plasma concentrations of total cholesterol and HDL cholesterol (mean± standard deviation) in the control mice (open squares) or after injection of 1×10⁹ pfu of adeno AdCMV hLCAT (open rings) or alternatively 1×10⁹ pfu of adeno AdCMV βgal (filled squares) in transgenic mice expressing the human apolipoprotein A-I.

*: various mice infected with the adeno AdCMV βgal, P<0.0001.

FIG. 6: Effect of the transfer of the human LCAT gene on the plasma concentrations of human apoA-I. Plasma concentrations of human apoA-I (mean±standard deviation) in the control mice (open squares) or after injection of 1×10⁹ pfu of adeno AdCMV hLCAT (open rings) or alternatively 1×10⁹ pfu of adeno AdCMV βgal (filled squares) in transgenic mice expressing the human apolipoprotein A-I.

*: various mice infected with the adeno AdCMV βgal, P<0.0001.

FIG. 7: Effect of the transfer of the human LCAT gene on the lipoprotein distribution of cholesterol. The plasmas derived from mice, 5 days after the injection of 5 10⁸ pfu of adeno AdCMV hLCAT (filled squares), 1×10⁹ pfu of adeno AdCMV hLCAT (solid rings) or controls (open squares). the plasma is separated on a Superose-6 column by gel-filtration chromatography and the cholesterol measured in each of the eluted fractions.

FIG. 8: Effect of the transfer of the human LCAT gene on the sizes of the EDL particles. The plasmas are obtained from mice, 5 days after the injection of 1×10⁹ pfu of adeno AdCMV hLCAT (solid line) and controls (dotted line). The plasmas were separated on a polyacrylamide gel (4-20% gradient) and transferred by Western blotting and the human apoA-I is then revealed by specific anti-human apoA-I antibodies. The blot is then scanned by densitometry.

FIG. 9: Effect of the transfer of the human LCAT gene on the mobility of the particles containing apoA-I. The plasmas are obtained from mice, 5 days after the injection of 1×10⁹ pfu of adeno AdCMV βgal (1), 5×10⁸ pfu of adeno AdCMV hLCAT (2) or 1×10⁹ pfu of adeno AdCMV hLCAT (3). 2 μl of plasma are used to separate the HDLs by agarose gel electrophoresis followed by staining of the lipids with Sudan black.

FIG. 10: Effect of the transfer of the human LCAT gene on the capacity of the serum to promote effluxes of cholesterol. The plasmas are obtained from mice, 5 days after the injection of 1×10⁹ pfu of adeno AdCMV hLCAT (open circles), 1×10⁹ pfu of adeno AdCMV βgal (solid squares) or control mice (open squares). The efflux of cholesterol is calculated by measuring the radioactivity in the medium and in the cells after incubating serum diluted to 2.5% with Fu5Ah cells precharged with radioactive cholesterol.

*: various control mice, P<0.01. **: various control mice or mice infected with the adeno AdCMV Agal, P_{—}0.0005.

### GENERAL MOLECULAR BIOLOGY TECHNIQUES

The methods conventionally used in molecular biology, such as preparative extractions of plasmid DNA, centrifugation of plasmid DNA in caesium chloride gradient, agarose or acrylamide gel electrophoresis, purification of DNA fragments by electroelution, phenol or phenol-chloroform extraction of proteins, ethanol or isopropanol precipitation of DNA in saline medium, transformation in Escherichia coli and the like, are well known to persons skilled in the art and are widely described in the literature [Maniatis T. et al., “Molecular Cloning, a Laboratory Manual”, Cold Spring Harbor Laboratory, Cold Spring Harbor, N.Y., 1982; Ausubel F. M. et al. (eds), “Current Protocols in Molecular Biology”, John Wiley & Sons, New York, 1987].

The pBR322 and pUC type plasmids and the phages of the M13 series are of commercial origin (Bethesda Research Laboratories).

For the ligations, the DNA fragments can be separated according to their size by agarose or acrylamide gel electrophoresis, extracted with phenol or with a phenol/chloroform mixture, precipitated with ethanol and then incubated in the presence of phage T4 DNA ligase (Biolabs) according to the recommendations of the supplier.

The filling of the protruding 5′ ends can be performed with the Klenow fragment of E. coli DNA polymerase I (Biolabs) according to the specifications of the supplier. The destruction of the protruding 3′ ends is performed in the presence of phage T4 DNA polymerase (Biolabs) used according to the recommendations of the manufacturer. The destruction of the protruding 5′ ends is performed by a controlled treatment with S1 nuclease.

Site-directed mutagenesis in vitro by synthetic oligodeoxynucleotides can be performed according to the method developed by Taylor et al. [Nucleic Acids Res. 13 (1985) 8749-8764] using the kit distributed by Amersham.

The enzymatic amplification of the DNA fragments by the so-called PCR technique [Polymerase-catalyzed Chain Reaction, Saiki R. K. et al., Science 230 (1985) 1350-1354; Mullis K. B. and Faloona F. A., Meth. Enzym. 155 (1987) 335-350] can be performed using a DNA thermal cycler (Perkin Elmer Cetus) according to the specifications of the manufacturer.

The verification of the nucleotide sequences can be performed by the method developed by Sanger et al. [Proc. Natl. Acad. Sci. USA, 74 (1977) 5463-5467] using the kit distributed by Amersham.

### EXAMPLES

### Example 1

### Construction of a Defective Recombinant Adenovirus Containing the Human Lecithin-Cholesterol Acyltransferase (hLCAT) Gene

As indicated above, the defective recombinant adenoviruses were prepared by homologous recombination between an adenovirus and a plasmid carrying, inter alia, the gene which it is desired to insert, after cotransfection into an appropriate cell line.

A. Preparation of the Plasmids Carrying the Human LCAT Gene

1. Construction of the Plasmid pXL2616

The plasmid pXL2616 contains the CDNA encoding human lecithin-cholesterol acyltransferase.

It was constructed in the following manner:

The DNA fragment corresponding to the LCAT cDNA was isolated by the RT-PCR technique from the total RNAs of the HepG2 cells (First-Strand cDNA synthesis Kit, Pharmacia). The cDNAs were produced by reverse transcription of the polyadenylated RNAs with the aid of hexanucleotide primers. A PCR reaction was then performed on these cDNAs with the oligonucleotides Sq5209 : CCC TCG AGG CCA TCG ATG AGG CCT GAC TTT TTC AAT AAA (SEQ ID No.1) and Sq5287 : GCG TCG ACA GCT CAG TCC CAG GCC TCA GAC GAG (SEQ ID No.2) which are specific for the human LCAT sequence (MacLean et al., Proc. Natl. Acad. Sci., 83, 1986) and which allow the addition of a ClaI site in 5′ of the LCAT sequence and of an SalI site in 3′. The 1750 bp fragment obtained was cloned into the plasmid pCR-II (TA cloning Kit, Invitrogen) and its sequence verified. The resulting plasmid was called pXL2616.

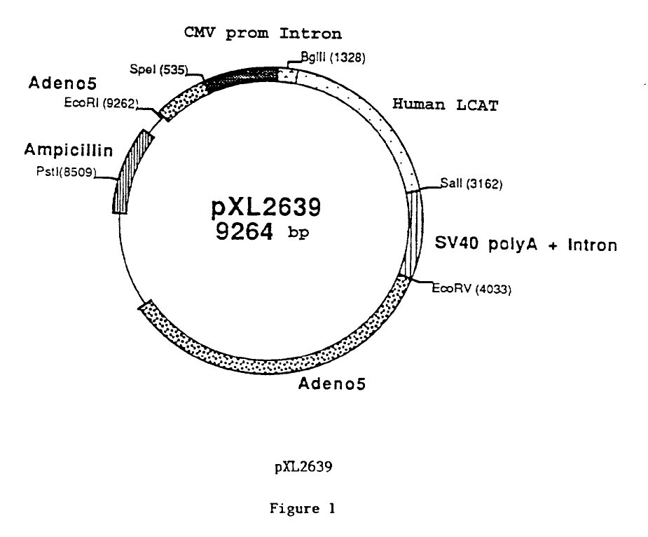

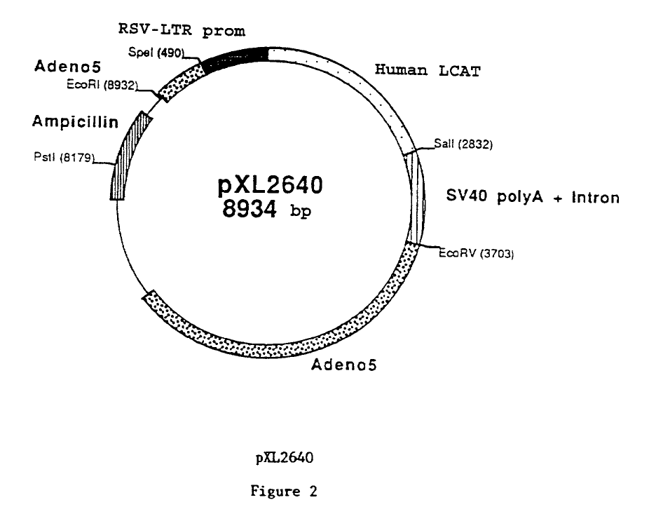

2. Construction of the Plasmids pXL2639 (FIG. 1) and pXL2640 (FIG. 2)

The plasmids pXL2639 and pXL2640 contain the human LCAT cDNA, under the control of the early CMV promoter and of the RSV virus LTR promoter respectively.

They were constructed in the following manner:

digestion of the plasmids pXL2375 (CMV promoter) and pXL2376 (RSV-LTR promoter), which are described in application WO 94/25073, with ClaI and SalI, which leads to the excision of the apoA-I cDNA, and then,

insertion of the ClaI-SalI fragment of the plasmid pXL2616, containing the human LCAT cDNA, into the previously digested plasmids described above.

B. Expression of the Human Lecithin-Cholesterol Acyltransferase in Vitro

The expression and the functionality of the enzyme were tested after transient transfection of cells 293 with the vectors thus constructed (pXL2639 and pXL2640). The DNA was introduced by means of a calcium phosphate-DNA complex according to the method of Wilger et al., Cell, 11 (1977) 223.

The LCAT activity was measured on the cellular supernatants 60 hours after the transfection, according to the Chen and Albers method, JLR, 23 (1982) 680. The measurement is based on the use of proteoliposomes as exogenous substrate, which are prepared by incubating for 30 minutes apoA-I 14C cholesterol, phosphatidylcholine at a molar ratio of 0.8:12.5:250 at 37° C. The activity is determined by measuring the conversion of 14C-cholesterol to 14C-cholesterolester after incubating the substrate with 4 μl of plasma or of culture supernatant for 2 hours at 37° C. The esters formed are separated by thin-layer chromatography on silica plates with the aid of a petroleum ether-diethyl ether-acetic acid mixture 76:20:1 and the radioactivity is determined by liquid scintillation spectrometry.

The results obtained show that the human LCAT secreted by the transfected cells 293 is functional.

C. Preparation of the Recombinant Adenoviruses

The plasmids prepared in A were then linearised and cotransfected for recombination with the deficient adenoviral vector, into the helper cells (line 293) which provide in trans the functions encoded by the adenovirus E1 regions (E1A and E1B).

The adenovirus Ad.CMVLCAT was obtained by homologous recombination in vivo between the adenovirus Ad.RSVβgal (Stratford-Perricaudet et al., J. Clin. Invest 90 (1992) 626) and the plasmid pXL2639 according to the following procedure: the plasmid pXL2639, linearised by the enzyme XmnI, and the adenovirus Ad.RSVβgal, linearised by ClaI, are cotransfected into the line 293 in the presence of calcium phosphate in order to allow the homologous recombination. The recombinant adenoviruses thus generated are selected by plaque purification. After isolation, the recombinant adenovirus is amplified in the cell line 293, which leads to a culture supernatant containing the unpurified recombinant defective adenovirus having a titre of about 10¹⁰ pfu/ml.

The viral particles are purified by caesium chloride gradient centrifugation according to known techniques (see especially Graham et al., Virology 52 (1973) 456). The adenovirus Ad.CMVLCAT is stored at −80° C. in 20% glycerol.

The same procedure was repeated with the plasmid pXL2640, leading to the recombinant adenovirus Ad.RSVLCAT.

### Example 2

### Expression in Vitro of the Human LCAT Gene Mediated by a Defective Recombinant Adenovirus

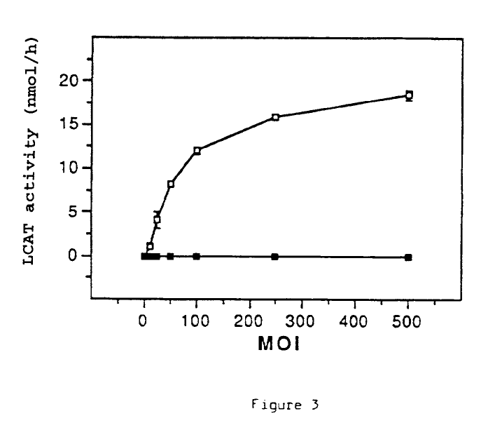

The expression and the functionality of the enzyme were tested after infection of Hep3B cells (human hepatocyte cell line) with the recombinant adenovirus AdCMV-hLCAT at MOIs of 10, 25, 50, 100, 250 and 500. The recombinant adenovirus AdCMVβgal was used as control. The LCAT activity (total quantity of cholesterol esters produced in 1 hour in 100 μl of culture medium) was measured on the cellular supernatants 72 hours after the infection, according to the method of Chen and Albers, JLR, 23 (1982) 680. The results (FIG. 3) show that the human LCAT secreted into the culture medium is functional and that the level of expression of the enzyme depends on the viral concentration in the cells.

### Example 3

### Expression in Vivo of the Human LCAT Gene Mediated by a Defective Recombinant Adenovirus

C57B1/6 mice transgenic for human apoA-1 were infected by injection into the vein of the tail of recombinant adenovirus AdCMV-hLCAT (5×10⁸ or 1×10⁹ pfu), AdCMV-βgal (1×10⁹ pfu) or of nonviral solution. Very high levels of LCAT activity were detected in the plasma of mice infected with AdCMV-hLCAT (from 3266±292 to 9068±812 nmol/ml/h), 5 days after the injection, whereas the levels observed in the mice not infected or infected with AdCMV-βGal correspond to the basal LCAT activity of the mouse plasma.

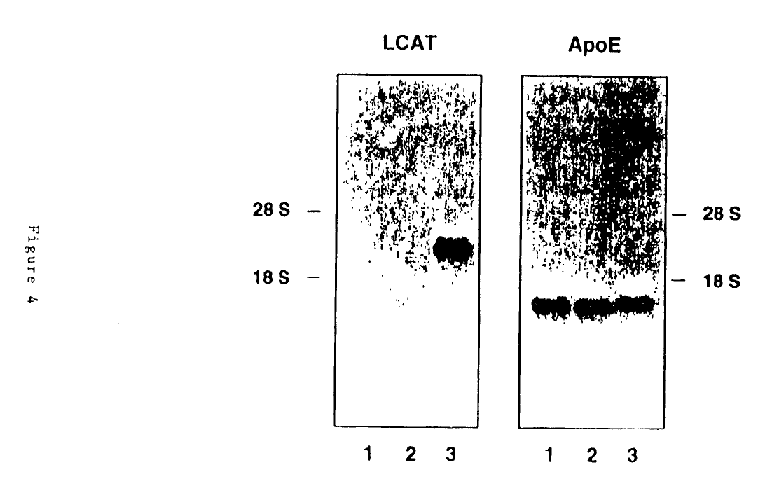

Northern blotting, carried out with the RNAs from the liver of mice infected with AdCMV-hLCAT made it possible to reveal the expression of only one species of messenger RNA which hybridizes with a probe corresponding to the complete cDNA for the human LCAT, whereas a Northern blotting carried out with the RNAs from the liver of the control mice showed no hybridization (FIG. 4).

### Example 4

### Effects of the Expression of Human LCAT on the Plasma Levels of the Lipoproteins and Apolipoproteins

The transient expression of the human LCAT caused a significant change in the concentrations of circulating lipids and of human apolipoprotein A-I (hapoA-I). The highest variations were observed 5 days after the injection and are summarized in Table I.

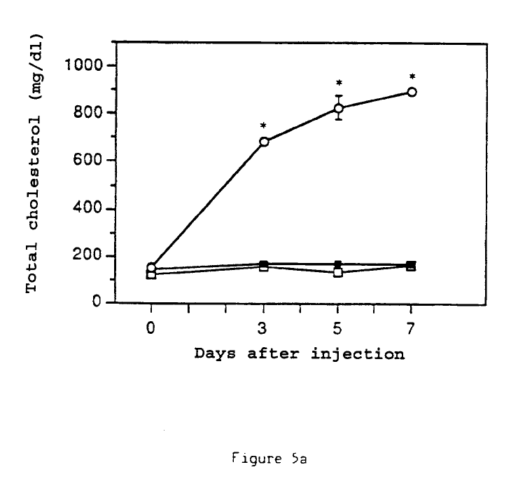

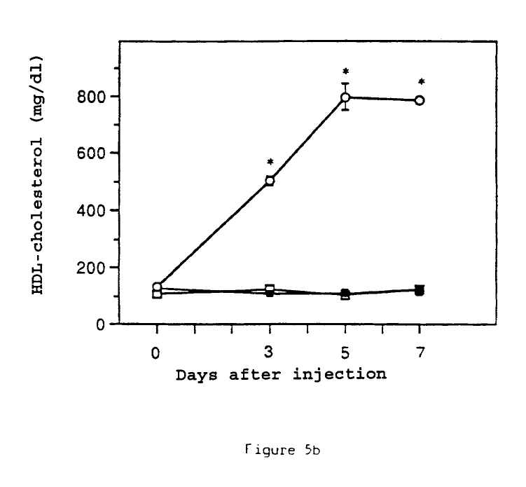

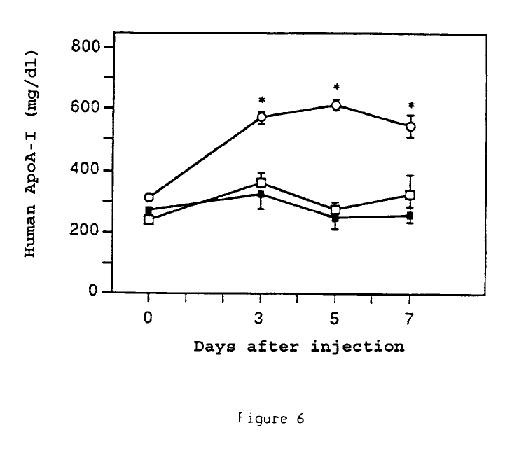

The mice infected with 1×10⁹ pfu of AdCMV-hLCAT have plasma levels of HDL-cholesterol and of total cholesterol (TC) 7 and 6 times greater, respectively, than the levels obtained in the control mice (FIG. 5a and 5b). These variations are associated with an increase both in the esterified cholesterol (EC) and in the free cholesterol (FC), respectively from 8 to 2.5 times compared with the levels obtained in the control mice. The increase in the plasma EC leads to an increase in the EC/TC ratio in the HDL fraction. The mice infected with 1×10⁹ pfu of AdCMV-hLCAT attribute a 2.5-fold increase in the concentration of human apoA-I compared with the control mice (FIG. 6).

### Table I. Lipid and Apolipoprotein Parameters in the Plasma of Control and Adenovirus-Infected Human apoA-I Transgenic Mice

TABLE I
Lipid and apolipoprotein parameters in
the plasma of control and adenovirus-infected
human apoA-I transgenic mice.

| | | Mice | Mice | |
|---|---|---|---|---|
| | | infected | infected | Mice |
| | | with | with | infected |
| | | AdCMV | AdCMV- | with AdCMV- |
| | | βgal | LCAT | LCAT |
| | Control | (n = 5) | (n = 2) | (n = 5) |
| | mice | 1 × 10⁹ | 5 × 10⁸ | 1 × 10⁹ |
| | (n = 5) | pfu/mice | pfu/mice | pfu/mice |
| Total | 132 ± 14 | 139 ± 18 | 462 ± 116^{c} | 827 ± 49^{a} |
| cholesterol | | | | |
| (TC) | | | | |
| Esters of | 68 ± 8 | 71 ± 10 | 319 ± 22^{b} | 587 ± 41^{a} |
| cholesterol | | | | |
| (EC) | | | | |
| Free | 63 ± 11 | 68 ± 9 | 143 ± 37^{c} | 239 ± 62^{b} |
| cholesterol | | | | |
| (FC) | | | | |
| EC/TC | 0.52 ± 0.06 | 0.51 ± 0.07 | 0.69 ± 0.04^{c} | 0.71 ± 0.04^{c} |
| (VLDL + | 15 ± 3 | 20 ± 6 | 33 ± 12^{d} | 30 ± 3^{c,e} |
| LDL) − | | | | |
| TC | | | | |
| Triglycer- | 49 ± 3 | 50 ± 7 | 90 ± 5^{c} | 140 ± 7^{b} |
| ides | | | | |
| Phospho- | 313 ± 40 | 309 ± 20 | 773 ± 53^{c} | 954 ± 65^{b} |
| lipids | | | | |
| h apoA-I | 247 ± 14 | 246 ± 30 | 542 ± 32^{c} | 616 ± 17^{a} |
| LCAT act- | 45 ± 2 | 45 ± 3 | 3266 ± 292^{a} | 9068 ± 812^{a} |
| ivity | | | | |
| (nmol/ml/ | | | | |
| h) | | | | |
| Endogen- | 149 ± 11 | 161 ± 17 | ND | 340 ± 5^{c} |
| ous ester- | | | | |
| ification | | | | |
| rate | | | | |
| (nmol/ml/ | | | | |
| h) | | | | |
| HDL-TC | 117 ± 12 | 119 ± 14 | 429 ± 127^{c} | 797 ± 48^{a} |
| HDL-EC | 66 ± 8 | 67 ± 10 | 317 ± 11^{b} | 570 ± 20^{a} |
| HDL-FC | 51 ± 11 | 52 ± 12 | 112 ± 26^{c} | 227 ± 53^{b} |
| EC/TC in | 0.56 ± 0.05 | 0.57 ± 0.05 | 0.74 ± 0.03^{c} | 0.72 ± 0.03^{c} |
| the HDLs | | | | |

All the lipid and lipoprotein values are expressed in mg/dl. ^{a}p<0.0001, ^{b}p<0.0004, ^{c}p<0.01, ^{d}p=NS. Different from the control mice and the mice infected with the adeno-AdCMV βgal-infected. ^{a}p=NS different from mice infected with the adeno AdCMV βgal-infected.

### Example 5

### Effects of the Expression of the Human LCAT on the Distribution of Cholesterol in the Lipoproteins, the Size and the Electrophoretic Mobility of the EDLs

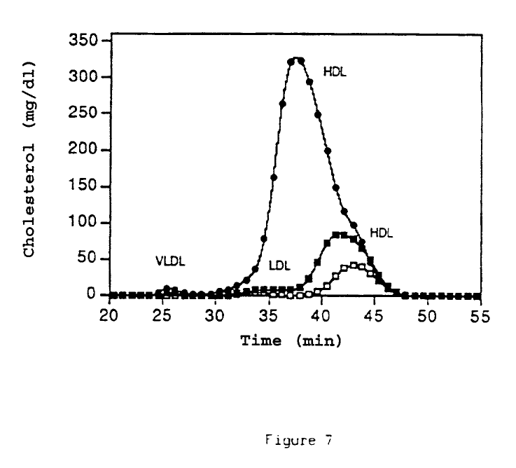

The distribution of cholesterol in the lipoprotein fractions was achieved using pools of plasmas from mice by analytical gel filtration chromatography (FIG. 7). The TC and human apoA-I concentrations were determined in the eluted fractions. These analyses reveal a substantial accumulation of cholesterol in the HDL fraction as well as an increase in the size of the HDLs for the mice infected with 1×10⁹ pfu of AdCMV-hLCAT compared with the control mice. The human apoA-I is found associated with the particles of the size of the HDLs.

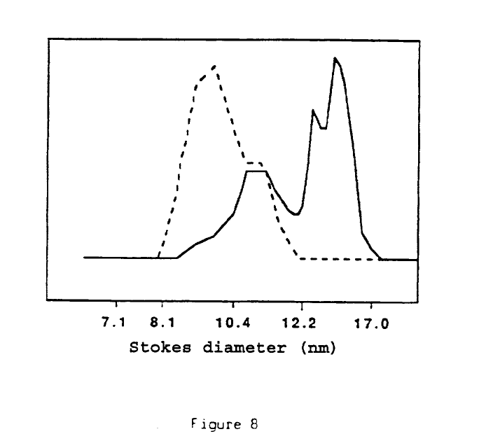

It was shown that the size distribution of the lipoproteins containing the apoA-I in the mice transgenic for human apoA-I was bimodal, with peak sizes of 9.4 nm and 11 nm. Whereas, in the control mice, this same distribution is conserved, it is altered in the mice infected with the AdCMV-hLCAT. For the mice which have received 1×10⁹ pfu of AdCMV-hLCAT, the smallest peak disappears in favour of two larger peaks of 13.3 and 14.2 nm (FIG. 8).

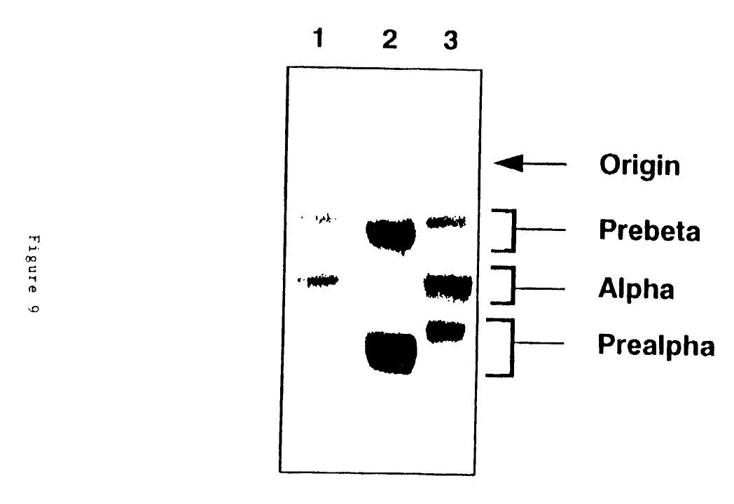

The plasma lipoproteins were separated by electrophoresis on a non-denaturing agarose gel, followed by detection of the lipids. As shown in FIG. 9, the HDLs having a pre-alpha mobility appear in the plasmas of the mice infected with AdCMV-hLCAT, revealing that not only is the size of the HDLs affected but also the charges at the surface of the HDLs.

In short, the high and transient expression of the human LCAT in mice transgenic for human apoA-I leads to the formation of a less atherogenic lipoprotein profile by virtue of the increase in the HDL-cholesterol and human apoA-I concentrations, as well as the increase in the HDL size and charge.

### Example 6

### Effects of the Expression of Human LCAT on the Efflux of Cellular Cholesterol

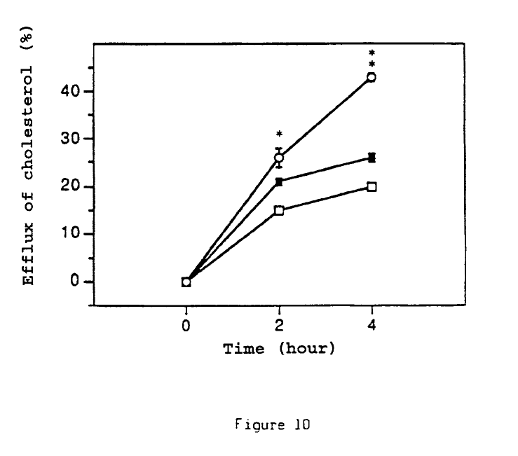

The efflux of cellular cholesterol was determined after incubation of rat hepatoma cells Fu5AH with pools of plasmas from infected or noninfected mice. FIG. 10 shows that a 65% increase in efflux is obtained with the plasma of mice infected with AdCMV-hLCAT compared with the plasma of mice infected with AdCMV βgal. It was found that this increase is in relation with the higher concentrations of human apoA-I and of HDL-cholesterol in the mice infected with AdCMV-hLCAT. These results support a higher efficiency in the reverse transport of the cholesterol resulting from the high expression of human LCAT.

1. Defective recombinant virus containing at least one inserted gene encoding all or part of lecithin-cholesterol acyltransferase (LCAT) or a variant thereof.

2. Virus according to claim 1, characterized in that it lacks the regions of its genome which are necessary for its replication in the infected cell.

3. Virus according to claim 1 or 2, characterized in that it is an adenovirus, preferably of the Ad 5 or Ad 2 type.

4. Virus according to claim 1 or 2, characterized in that it is an adenovirus of animal, preferably canine, origin.

5. Virus according to one of claims 1 to 4, characterized in that the inserted gene encodes all or part of human LCAT or a variant thereof.

6. Virus according to claim 5, characterized in that the inserted gene encodes human LCAT.

7. Virus according to one of claims 1 to 6, characterized in that the inserted gene is a cDNA.

8. Virus according to one of claims 1 to 6, characterized in that the inserted gene is a gDNA.

9. Virus according to one of claims 1 to 8, characterized in that the inserted gene comprises sequences allowing its expression in the infected cell.

10. Virus according to one of claims 1 to 9, characterized in that the inserted gene comprises a signal sequence directing the synthesized polypeptide in the secretory pathways of the target cell.

11. Adenovirus according to claim 3 or 4, characterized in that it comprises a deletion of all or part of the E1 region.

12. Adenovirus according to claim 11, characterized in that it comprises, in addition, a deletion of all or part of the E4 region.

13. Virus according to claim 1 or 2, characterized in that it is an adeno-associated virus (AAV).

14. Virus according to claim 13, characterized in that its genome comprises the gene encoding all or part of lecithin-cholesterol acyltransferase (LCAT) or a variant thereof, bordered by 2 ITRs.

15. Virus according to claim 1 or 2, characterized in that it is a retrovirus.

16. Use of a virus according to one of claims 1 to 15, for the preparation of a pharmaceutical composition intended for the treatment or the prevention of pathologies linked to dyslipoproteinaemias.

17. Use according to claim 16, for the preparation of a pharmaceutical composition intended for the treatment of atherosclerosis and/or of restenosis.

18. Pharmaceutical composition comprising one or more defective recombinant viruses according to one of claims 1 to 15.

19. Pharmaceutical composition according to claim 18, characterized in that it is provided in an injectable form and in that it comprises from 10⁴ to 10¹⁴ pfu/ml of adenovirus.

20. Pharmaceutical composition according to claim 19, characterized in that it also contains one or more defective recombinant adenoviruses encoding an apolipoprotein.
